(19) United States
(12) Patent Application Publication
(10) Pub. No.: US 2004/0208781 A1
(43) Pub. Date: Oct. 21, 2004

(54) Article sterilizing methods and sterilizing device

(76) Inventors: Makoto Hayashi, Tokyo-to (JP); Atsushi Hayakawa, Tokyo-to (JP); Masatoshi Takagi, Tokyo-to (JP); Akira Koyama, Tokyo-to (JP)

Correspondence Address: LADAS & PARRY, 224 SOUTH MICHIGAN AVENUE, SUITE 1200, CHICAGO, IL, 60604, US

(21) Appl. No.: 10/488,712

(22) PCT Filed: Sep. 5, 2002
(86) PCT No.: PCT/JP02/09045

(30) Foreign Application Priority Data
Sep. 5, 2001 (JP) 2001-269417

(51) Int. Cl. A61L 2/20

(52) U.S. Cl. 422/28; 422/302; 422/303

(57) ABSTRACT

An apparatus for sterilizing the inside of an article such as a PET bottle comprises a hot air supplying device (14) for supplying hot air to the inside of an article (1), and a mist generating device (33) for producing mists of a sterilizing agent. By mixing the mists produced by the mist generating device with the hot air supplied by the hot air supplying device and introducing the same to the inside of the article, the sterilizing agent mists can be introduced evenly to every corner in the inside of the article so as to obtain an even sterilizing effect. Furthermore, the sterilizing apparatus comprises an air rinsing device (56) for discharging the air including the mists inside the article by blowing in the sterilized air to the inside of the article, and a washing device (57) for supplying a washing agent to the inside of the article.

### TECHNICAL FIELD

The present invention relates to a method for sterilizing the article such as a PET bottle, and a sterilizing apparatus.

### BACKGROUND ART

As a sterilizing method for use in a sterile filling system for drink bottles, a method of introducing into bottles mists of a hydrogen peroxide as a sterilizing agent for sterilization after raising the temperature of the bottles by blowing in hot air into the bottle inner surface is known (see Japanese Patent Application Laid-Open (JP-A) No. 2001-39414).

According to the conventional method, since the sterilizing agent mists are jetted from a nozzle disposed immediately above the bottles conveyed on a line, a large amount of the hydrogen peroxide should be supplied from the nozzle in order to adhere the mists evenly on the inner surface of the bottle. Moreover, since the sterilizing effect by the mists largely depends on the temperature of the bottle, the even sterilizing effect cannot be obtained in the case where irregularity is generated in the bottle temperature distribution after the preliminary heating operation. Furthermore, since the preliminary heating operation for the bottle and the mist introduction are executed separately, for sufficiently contacting the bottles and the mists, the bottle preliminary heating operation needs to be finished in a short time. Therefor, the temperature and the flow rate of the hot air to be supplied to the bottles must inevitably be set higher, and as a consequence, there is a risk of heating the bottles at an unnecessarily high temperature. In particular, since resin bottles such as PET bottles are weak to the heat, there is a risk of deforming the same by the high temperature heating operation.

### DISCLOSURE OF THE INVENTION

The present invention has been achieved in view of the above-mentioned circumstances, and an object thereof is to provide a sterilizing method and apparatus capable of obtaining the even and excellent sterilizing effect by evenly introducing mists of a sterilizing agent such as a hydrogen peroxide into the article while heating the article at an appropriate temperature.

A sterilizing method according to the present invention comprises a process of mixing mists of a sterilizing agent with hot air, and a process of supplying the hot air mixed with the mists into the inside of an article.

According to the method, the mists of the sterilizing agent can be introduced to every corner of the inside of the article evenly by utilizing the hot air supplied into the article. Since the temperature of the inside of the bottle can be maintained constantly by supplying the heat into the bottle continuously during the mist introducing operation, even in the case where the hot air temperature and flow rate are set lower, the even and sufficient sterilizing effect can be obtained. That is, according to the present invention, since the sterilizing condition of introducing the mists of the sterilizing agent at a constant temperature for a certain period into the inside of the article can be realized easily, the even and excellent sterilizing effect can be obtained certainly.

The sterilizing method of the present invention may comprise a process of washing the inside of the article with a washing agent after supplying the hot air mixed with the mists into the inside of the article. By washing the inside of the article with the washing agent, the remaining mists of the sterilizing agent can be prevented certainly.

The sterilizing method of the invention may comprise a process of discharging the air containing the mists inside the article by blowing in the sterilized air into the inside of the article after supplying the hot air mixed with the mists into the inside of the article, and a process of washing the inside of the article with a washing agent after discharging the air including the mists. In this case, by blowing in the air into the inside of the article so as to discharge the mists of the sterilizing agent, progress of adsorption and permeation of the sterilizing agent component into the article can be prevented. Thereby, the washing effect of the article inside can be improved. In particular, in the case where the time from stopping the introduction of the mists to the start of the supply of the washing agent is longer than the tolerance range in view of the sterilizing agent component adsorption or permeation due to the washing preparation convenience, or the like, it is effective to discharge the mists by blowing the sterilized air prior to the washing operation. As the washing agent, the sterilized water can be utilized preferably, however, in addition thereto, as long as there is no risk of having a substance not preferable in view of the article application remaining, various liquids can be used as the washing agent.

In the process of supplying the hot air mixed with the mists, it is possible that the hot air is blown in from a nozzle by inserting a nozzle into the inside of the article, and a guiding member is disposed around the nozzle outside the article so that the hot air discharged from the article is guided to the outer surface side of the article by the guiding member. According to the method, the outer surface in the vicinity of an opening in which the nozzle of the article is inserted, for example in the case of a bottle, the outer surface of its mouth part can be sterilized efficiently by utilizing the mists introduced into the inside of the article. As the guiding member, one capable of guiding the hot air including the mists to the outside of the article so as to flow along the outer surface of the article can be used, and the shape, the arrangement, and the number are not limited.

In the washing process, the washing efficiency can be improved by heating the washing agent to be supplied into the article. Moreover, in the process of discharging the air including the mists, by heating the sterilized air and blowing the same into the article, the adsorption and the permeation of the sterilizing agent component into the article can be restrained so that the washing effect in the subsequent washing process can be improved.

A sterilizing apparatus of the present invention comprises a hot air supplying device for supplying hot air to the inside of an article, and a mist generating device for producing mists of a sterilizing agent, wherein the mists produced by the mist generating device are introduced to the inside of the article while being mixed with the hot air supplied by the hot air supplying device.

According to the apparatus, by mixing the mists of the sterilizing agent with the hot air to be introduced to the inside of the article, the even and excellent sterilizing effect can be obtained certainly by realizing the sterilizing method of the present invention.

As in the sterilizing method of the present invention, the sterilizing apparatus of the present invention can also include the following aspects.

That is, the sterilizing apparatus of the present invention may comprise a washing device for supplying a washing agent to the inside of the article The sterilizing apparatus of the present invention may comprise an air rinsing device for discharging the air including the mists inside the article by blowing in the sterilized air to the inside of the article, and a washing device for supplying a washing agent to the inside of the article.

The hot air supplying device may comprise a nozzle to be inserted to the inside of the article for blowing in the hot air into the article, and a guiding member provided so as to surround the nozzle at the outside of the article for guiding the hot air discharged from the article to the outer surface side of the article.

The washing device may heat the washing agent and supply the same to the inside of the article. The air rinsing device may heat the sterilized air and blow in the same into the article.

In the present invention, the article may be a food container, or have a bottle shape. In the present invention, the mists of the sterilizing agent denote minute spray obtained by temporarily vaporizing the sterilizing agent droplets to the boiling point or higher and quenching. In the present invention, the concept of the germless is not limited to the state of complete absence of the germs, and as long as it is the state with the number of the germs present is reduced to the tolerance range determined according to the application of the article, or the like, it can be included substantially in the sterile state. Also as to the concept of the sterilization, as long as the germs can be reduced to the sterilized state, it is included in the range.

### BRIEF DESCRIPTION OF THE DRAWINGS

FIG. 1 is a view showing the procedure of a sterilizing method in a first embodiment of the present invention;

FIG. 2 is a diagram showing a device for mixing hot air with sterilizing agent mists and supplying the same into a bottle;

FIG. 3 is a view showing the schematic configuration of a mist generating device;

FIG. 4 is a view showing the procedure of a sterilizing method in a second embodiment of the present invention;

FIG. 5 is a view showing a hot air supplying device with a guiding member mounted;

FIG. 6 is a diagram showing an example of a sterile filling system to which the sterilizing method of the present invention is applied; and

FIG. 7 is a view showing a mist introducing device provided in the sterile filling system of FIG. 6.

### BEST MODE FOR CARRYING OUT THE INVENTION

(First Embodiment)

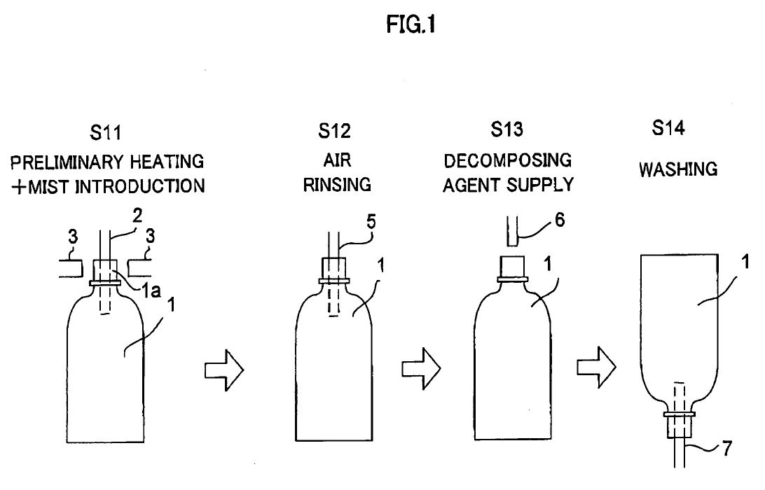

FIG. 1 is a view showing the procedure of a sterilizing method according to a first embodiment of the present invention. This embodiment shows a method of sterilizing the inner surface of a PET (polyethylene terephthalate) bottle. In this sterilizing method, first, a preliminary heating process is executed (step S11). In the preliminary heating, a nozzle 2 is inserted to the inside from a mouth part 1a of a bottle 1 so that hot air is sent from the nozzle 2 so as to preliminarily heat the bottle 1. Simultaneously, nozzles 3, 3 are placed on the outer circumference of the mouth part 1a of the bottle 1 so that the hot air is blown to the mouth part 1a from the nozzles 3 so as to further heat the mouth part 1a. Moreover, at the same time with the preliminary heating process, a process of introducing mists of a sterilizing agent into the bottle 1 is executed as well. The mist introduction is realized by mixing the mists with the hot air to be supplied for the preliminary heating.

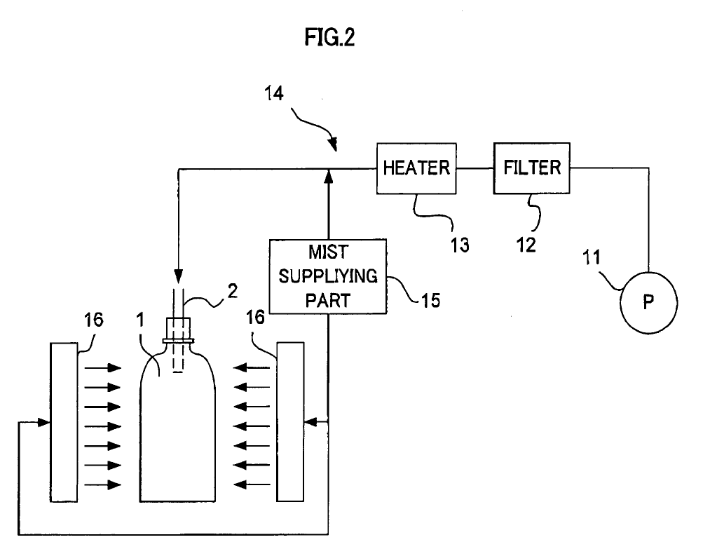

FIG. 2 shows a schematic configuration of an apparatus used for the preliminary heating and mist supplying processes. This apparatus is provided as a hot air supplying device 14 for preliminarily heating the bottle 1 by heating the air sent from a blower (or a pump) 11 with a heater 13 after filtration by a filter 12, and supplying the heated air from the nozzle 2 to the inner surface of the bottle 1. It is executed desirably with the preliminary heating temperature such that the bottle 1 inner surface becomes 40° C. or higher. The inner surface temperature of the bottle 1 in the preliminary heating is further desirably in a range of 55° C. to 60° C. The hot air provided from the heater 13 to the nozzle 2 is mixed with the mists of the sterilizing agent containing a hydrogen peroxide as the main component sent from a mist supplying part 15. Therefore, the hot air supplied from the nozzle 2 includes the sterilizing agent mists so that the bottle 1 is preliminarily heated and simultaneously, the inner surface thereof is sterilized with the sterilizing agent mists. Other nozzles 16 are placed around the bottle 1 so as to surround the same, and the sterilizing agent mists are sent to the nozzles 16 from the mist supplying part 15. Thereby, the outer surface of the bottle 1 is sterilized simultaneously.

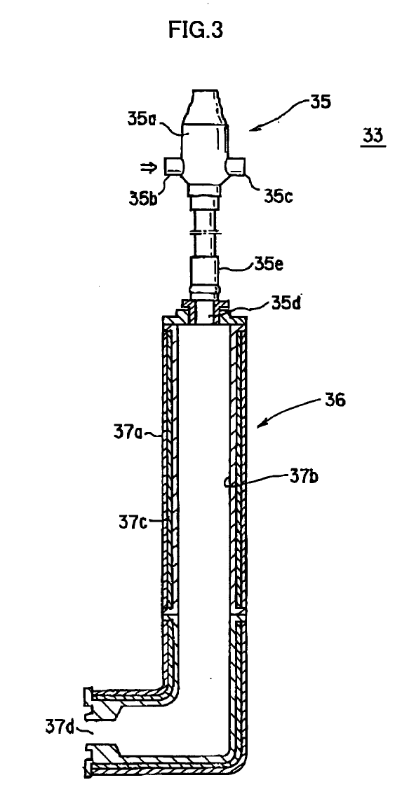

The mist supplying part 15 includes a mist generating device 33 shown in FIG. 3. The generating device 33 comprises a sterilizing agent supplying part 35 for making an aqueous solution of a hydrogen peroxide (H₂O₂) in droplets as the sterilizing agent and supplying the same, and a vaporizing part 36 for heating to its boiling point or higher and vaporizing the aqueous solution of the hydrogen peroxide supplied from the sterilizing agent supplying part 35. The sterilizing agent supplying part 35 is provided with a spray 35a. The spray 35a is provided with a sterilizing agent supply port 35b and a compressed air supply port 35c, with the ports 35b, 35c each connected with an unshown hydrogen peroxide supply source or jetting compressed air supply source.

By mixing the aqueous solution of the hydrogen peroxide and the compressed air supplied from the supply ports 35b, 35c inside the two-fluid spray 35a, the aqueous solution of the hydrogen peroxide is sprayed from a nozzle 35d connected with the spray 35a via an extension pipe 35e into a vaporizing pipe 37 of the vaporizing part 36. The vaporizing pipe 37 has, for example, an outer cylinder 37a comprising an asbestos ribbon, an inner cylinder 37b comprising a sanitary pipe for forming the inner wall of the vaporizing pipe 37, and a heater 37c as a heating device provided between the outer cylinder 37a and the inner cylinder 37b. A discharge port 37d at the lower end of the vaporizing pipe 37 is connected with the nozzle 2.

The droplet-like hydrogen peroxide supplied to the inside of the vaporizing pipe 37 is vaporized by the heat of the heater 37c. The vaporized hydrogen peroxide is liquefied and quenched by the temperature drop to the introduction to the vicinity of the bottle 1 via the nozzle 2. Thereby, mists of the hydrogen peroxide more minute than the droplets of the hydrogen peroxide produced by the two-fluid spray 35a can be produced By introducing the mists of the hydrogen peroxide to the inside of the bottle 1, the inner surface of the bottle 1 is contacted with the hydrogen peroxide so as to be sterilized.

The adhesion amount of the hydrogen peroxide mists with respect to a 500 ml capacity bottle can be 20 μl more based on a 35% by weight hydrogen peroxide solution, and it is preferably in a range of 20 μl to 100 μl. That is, it is preferable that the mist amount is set such that the hydrogen peroxide is adhered in the bottle 1 equivalently to the case of supplying the hydrogen peroxide solution containing a hydrogen peroxide by 35% by weight in a range of 20 μl to 100 μl. Moreover, in the case of a 2,000 ml capacity bottle, similarly, it can be 50 μl or more, and it is preferably in a range of 50 μl to 200 μl. The mist blowing time is preferably in a range of 0.1 second to 1 second with respect to one bottle. The concentration of the hydrogen peroxide contained in the produced mists is desirably 35% by weight or more. The sterilizing agent is not limited to the hydrogen peroxide, but various kinds of chemicals having the sterilizing function can be used as well.

Returning to FIG. 1, an air rinsing process is executed after supplying the mists (step S12) In the air rinsing process, the sterilized hot air is sent from the nozzle 5 in a state with the nozzle 5 inserted to the inside of the bottle 1 or without the insertion. The bottle 1 is heated by the hot air from the inner surface thereof so that the sterilizing effect by the sterilizing agent mists can be improved, and the adsorption and the permeation of the hydrogen peroxide to the bottle 1 are restrained so that the hydrogen peroxide can easily appear on the inner surface of the bottle 1. Furthermore, the mists flowing inside the bottle 1 can be discharged to the outside of the bottle 1 by the hot air. At this point, since the sterilization is carried out already sufficiently by the sterilizing agent mists adhered on the inner surface of the bottle 1, even in the case where the mists flowing in the internal space of the bottle 1 are discharged, the sterilizing effect cannot be deteriorated. Rather by discharging the excessive mists at the early stage, the excessive adsorption and permeation of the hydrogen peroxide to the inner surface of the bottle 1 can be restrained.

The hot air blowing-in operation can be executed in a range capable of discharging all the mists flowing inside the bottle 1. As to the time, about 1 second to 5 seconds is sufficient. In the case where the hot air temperature is equal to or higher than the heat resistant temperature of the bottle 1, if the hot air blowing-in time is too long, the bottle 1 is heated to above the heat resistant temperature so as to generate deformation, or the like, and thus it should be carried out carefully. Instead of the hot air, ordinary temperature air can be blown in for discharging the mists.

Subsequent to the air rinsing process, a decomposing agent supplying process is executed (step S13). In the decomposing agent supplying process, an aqueous solution of a decomposing agent containing a catalase as the main component is sprayed from the spray nozzle 6 so as to be supplied to the inside of the bottle 1. The concentration and the supply amount of the catalase aqueous solution can be selected optionally according to the hydrogen peroxide residual amount, the target rinsing time, or the like, however, the supply amount is set such that the predicted residual component of the hydrogen peroxide after the air rinsing operation can certainly be decomposed. For the mist supply amount, supply of 500 U/ml or more of the catalase aqueous solution is sufficient. The decomposing agent may be sprayed with the top end part of the nozzles 6 inserted to the inside of the bottle 1. As to the decomposing agent jetting method, from the viewpoint of adhering the aqueous solution rapidly and evenly to the inner surface of the bottle 1, it is carried out desirably by a one-fluid spray, a two-fluid spray, or a device having a scattering ability equivalent thereto.

Since the decomposing agent is added into the bottle after the bottle sterilization, naturally the decomposing agent itself must be sterilized. Moreover, the parts through which the decomposing agent passes, such as a spray piping for jetting the decomposing agent, must be sterilized preliminarily. In the case where a catalyst liquid (protein) is used as the decomposing agent, since the thermal sterilization cannot be carried out, the microbes are sterilized by filtration with a filter Moreover, the jetting spray and the piping can be sterilized by vapor sterilization (heat sterilization) or with a sterilizing agent.

After the supply of the decomposing agent, the bottle 1 is maintained for a predetermined time (for example, about 1 second to 5 seconds) and thereafter, a washing process is executed (step S14). In the washing process, the bottle 1 is inverted upside down, the nozzle 7 is inserted to the inside of the bottle 1, and the heated sterilized water is sent from the nozzle 7 as the washing agent. Thereby, the decomposing agent and an extremely minute amount of the hydrogen peroxide remaining in the inside of the bottle 1 can be washed out. The sterilized water may be at an ordinary temperature, however, it is preferable to heat the same for improving the washing efficiency. The washing agent temperature is desirably in a range of 40° C. to 80° C. As to the washing time, for example, for a 500 ml capacity bottle, it can be completed in about 3 seconds.

The decomposing agent may be supplied before the washing operation with the sterilized water. In the above-mentioned embodiment, the decomposing agent supplying process can be executed before the air rinsing process.

Accordingly, since the sterilization with the mists is executed simultaneously with the preliminary heating in this embodiment, the sterilizing process can be executed efficiently. Since the decomposing process using the decomposing agent is carried out, there main of the hydrogen peroxide can be restrained sufficiently. In this embodiment, the sterilizing agent mists supplied to the inside of the bottle can be discharged consecutively with the hot air for the preliminary heating. Therefore, the air rinsing process can be omitted.

(Second Embodiment)

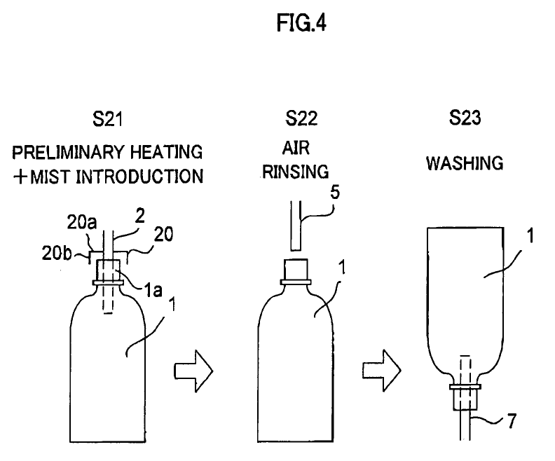

FIG. 4 is a view showing the procedure of a sterilizing method of a second embodiment of the present invention. This embodiment also has a PET bottle as the sterilization subject.

According to the sterilizing method of FIG. 4, first the preliminary heating process is executed (step S21). In the preliminary heating, the nozzle 2 is inserted from the mouth part 1a of the bottle 1 to the inside for sending in the hot air from the nozzle 2 so as to preliminarily heat the bottle 1, and with the preliminary heating process, a process of introducing the sterilizing agent mists into the bottle 1 is executed. The mist introduction can be realized by mixing the mists with the hot air for the preliminary heating by the device shown in FIG. 2.

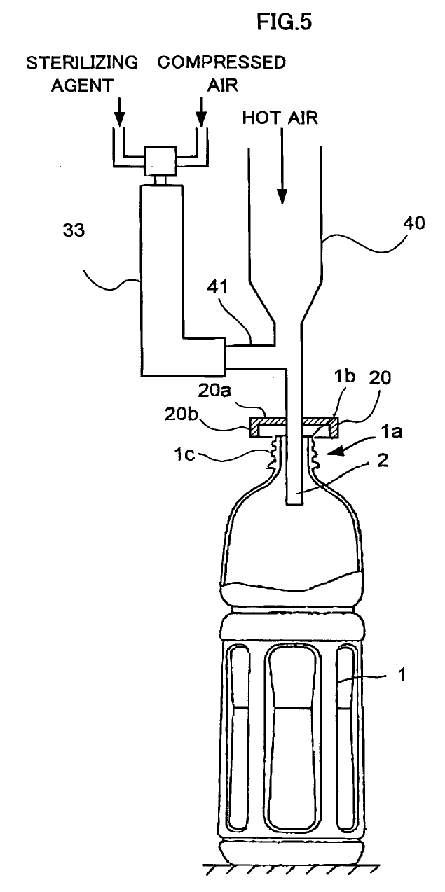

Moreover, according to the sterilizing method of FIG. 4, instead of the nozzle 3 shown in FIG. 1, the preliminary heating and the sterilization of the outer surface of the mouth part 1a of the bottle 1 are executed by utilizing a guiding member 20 provided to the nozzle 2. As it is shown in FIG. 5, the guiding member 20 is mounted to the nozzle 2 on the downstream side of a junction position of a duct 40 for the hot air and a duct 41 from the mist generating device 33. The guiding member 20 is provided with a flange part 20a provided concentrically with the nozzle 2, and a ring-like wall part 20b projecting from the outer circumference of the flange part 20a to the bottle 1 side. By mounting the guiding member 20 on the nozzle 2 and disposing the same in the vicinity of the mouth part la, the hot air blown from the mouth part 1a to the outside of the bottle can be guided to the outer circumference side of the mouth part 1a for blowing the hot air to a top surface 1b or a screw part 1c so as to execute the preliminary heating and the sterilization thereof. By utilizing the guiding member 20, regardless of whether or not the outer surface sterilization of the bottle 1 is executed by utilizing the nozzle 16 of FIG. 2, or the like, the boundary part of the outer surface and the inner surface of the bottle 1 can be sterilized efficiently and certainly.

In this embodiment, the flow rate of the hot air containing the sterilizing agent mists to be supplied to the inside of the bottle 1 is set in a range of 0.1 m³ to 0.8 m³, and preferably by 0.2 m³ to 0.3 m³ per minute. The hot air blowing-in time is preferably in a range of 2 seconds to 8 seconds. The amount of the hydrogen peroxide contained in the hot air to be supplied to the inside of the bottle 1 is preferably in a range of 2 mg to 6 mg for 1 L (liter) of the hot air.

Returning to FIG. 4, after continuing the preliminary heating and the mist introduction for a certain period, the air rinsing process is executed subsequently (step S22). In the air rinsing process, the sterilized hot air is sent from the nozzle 5 disposed immediately above the mouth part 1a of the bottle 1 into the bottle 1. In the same manner as in the first embodiment, the bottle 1 is heated from the inner surface by the hot air so that the sterilizing effect by the sterilizing agent mists can be improved, and the adsorption and the permeation of the hydrogen peroxide to the bottle 1 can be restrained so that the hydrogen peroxide can easily appear on the inner surface of the bottle 1. Furthermore, the mists flowing inside the bottle 1 are discharged to the outside of the bottle 1 by the hot air. At this point, since the sterilization is executed already sufficiently with the sterilizing agent mists adhered on the inner surface of the bottle 1, even in the case where the mists flowing in the internal space of the bottle 1 are discharged, the sterilizing effect cannot be deteriorated. Rather by discharging the excessive mists at the early stage, the excessive adsorption and permeation of the hydrogen peroxide to the inner surface of the bottle 1 can be restrained.

In this embodiment, the time for starting the hot air blowing-in operation after stopping the introduction of the sterilizing agent mists is desirably as short as possible. The time is set within 10 seconds at the longest, and desirably within 5 seconds. The hot air blowing-in operation can be executed in a range capable of discharging all the mists flowing inside the bottle 1, and as to the time, about 1 second to 5 seconds is sufficient. As to the temperature of the hot air, it is set desirably at a temperature as high as possible in a range without the risk of the deformation of the bottle 1 from the viewpoint of eliminating the hydrogen peroxide in the bottle 1. In the case of a PET bottle, the hot air temperature used for the air rinsing operation is set in a range of 50° C. or higher and lower than 150° C., and preferably in a range of 75° C. or higher and lower than 120° C. In the case where the hot air temperature is at the heat resistant temperature of the bottle 1 or higher, if the hot air blowing-in time is too long, the bottle 1 is heated to above the heat resistant temperature so as to generate deformation, or the like, and thus it should be carried out carefully. Instead of the hot air, ordinary temperature air can be blown in for discharging the mists.

As to the nozzle 5, it is desirable to insert the same to the inside of the bottle 1 for efficiently supplying the hot air. However, in the case where the mechanism for inserting the nozzle 5 into the bottle 1 is complicated, the air rinsing process can be executed with the nozzle 5 disposed outside the bottle 1.

After the air rinsing process, subsequently, the washing process for the inside of the bottle 1 with the sterilized water is executed (step S23). In the washing process, the bottle 1 is inverted upside down, the nozzle 7 is inserted to the inside of the bottle 1, and the heated sterilized water is sent from the nozzle 7 as the washing agent. Thereby, an extremely minute amount of the hydrogen peroxide remaining in the inside of the bottle 1 can be washed out. The sterilized water may be at an ordinary temperature, however, it is preferable to heat the same for improving the washing efficiency. The washing agent temperature is desirably in a range of 40° C. to 80° C. As to the washing time, for example, for a 500 ml capacity bottle, it can be completed in about 3 seconds.

In this embodiment, the air rinsing process may be omitted so that the washing operation can be executed subsequent to the preliminary heating and mist introduction processes. However, in the case where the time is required from the stoppage of the mist introduction to the start of the washing operation, it is preferable to provide the air rinsing process for restraining the adsorption and the permeation of the sterilizing agent.

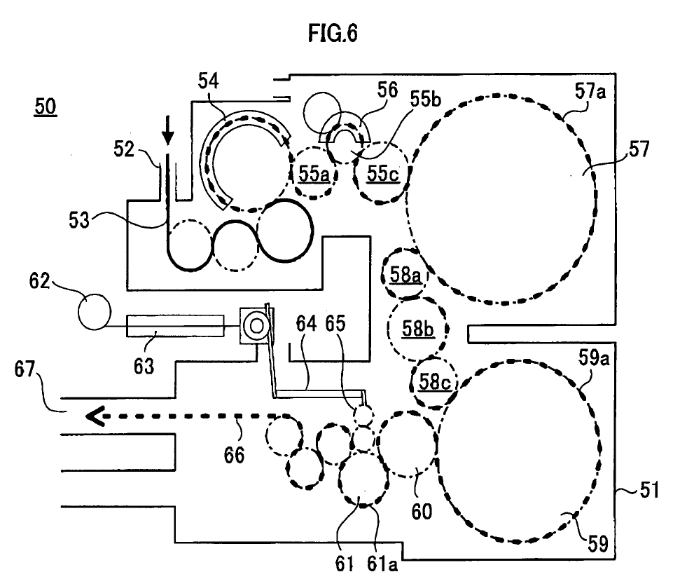

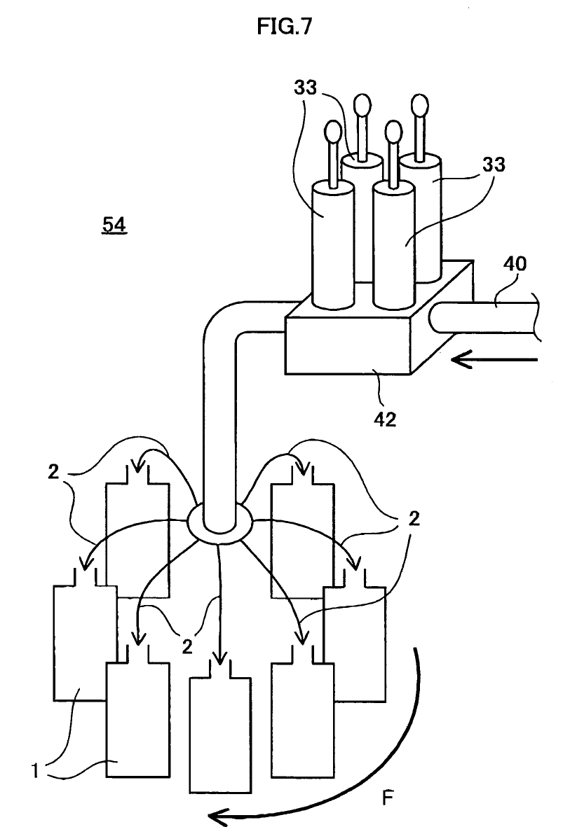

The above-mentioned sterilizing method can be applied for a sterile filling system of various configurations. Hereinafter, with reference to FIGS. 6 and 7, the sterile filling system capable of realizing the sterilizing method of the second embodiment will be explained. According to the sterile filling system 50 of FIG. 6, a bottle 1 introduced from an introduction port 52 of a sterile chamber 51 is guided to a mist introducing device 54 by a conveying line 53 so as to be subjected to the sterilizing process. In the mist introducing device 54, the mists of the hydrogen peroxide is jetted to the outer surface of the bottle 1 so as to sterilize the outer surface, and the inner surface of the bottle 1 is sterilized by the above-mentioned method. As it is shown also in FIG. 7, a plurality of nozzles 2, . . . 2 are provided in the mist introducing device 54 such that the nozzles 2 are moved in a predetermined direction (arrow F direction) along a round circulation path with the bottle 1 in a state inserted in the bottle 1. The moving rate of the bottle 1 in the mist introducing device 54 is constant, and it is also constant in the section with the nozzle 2 inserted in the bottle 1. Thereby, while supplying the hot air of a constant temperature to the inside of the bottle 1, the mists of the sterilizing agent are introduced only for a certain time.

According to the mist introducing device 54 of FIG. 7, by joining the hot air guided by the dust 40 and the mists supplied from a plurality of the mist generating devices 33, . . . 33 by a manifold 42 and distributing the same to each nozzle 2, the mists can be introduced from a large number of the nozzles 2 at one time.

Returning to FIG. 6, the bottle 1 passed through the mist guiding device 54 is guided to a turn table 57a of a washing device 57 after successively passing through turn tables 55a to 55c. The intermediate turn table 55b serves as a part of the air rinsing device 56. In the air rinsing device 56, by disposing the nozzle 5 (see FIGS. 1 and 4) above the bottle 1 being conveyed by the turn table 55b and moving the nozzle following the turn table 55b so as to maintain the same positional relationship with respect to the bottle 1 while sending the sterile air from the nozzle 5, the sterile air is blown into the bottle 1 for a constant time. The bottle 1 after passing through the air rinsing device 56 enters the washing device 57. The bottle 1 placed on the turntable 57a is inverted in the vertical direction by an inverting device (not shown) so that the nozzle 7 shown in FIGS. 1 and 4 is inserted to the inverted bottle 1, and the heated sterile water is sent from the nozzle 7 to the inside of the bottle 1 with the nozzle 7 following the movement of the bottle 1 for washing the inside of the bottle 1.

The bottle 1 washed by the washing device 57 is sent to a turn table 59a of a filling device 59 after passing through turn tables 58a to 58c. In the filling device 59, a predetermined content, such as a drink is filled to the inside of the bottle 1 while conveying the bottle 1 along the turn table 59a. The bottle 1 filled with the drink is sent to a turn table 61 a of a lid fastening device 61 via a turn table 60. In the lid fastening device 61, a cap taken out from a cap feeder 62 installed outside the sterile chamber 51 and sterilized by a cap sterilizing device 63 is supplied via a cap chute 64 and a turn table 65. The supplied cap is mounted on the bottle 1 by the lid fastening device 61 so as to seal the bottle 1. The bottle 1 after the sealing operation is taken out from a taking-out port 67 of the sterile chamber 51 by a conveying line 66 to the outside.

The present invention is not limited to the above-mentioned embodiment, and it can be carried out in various embodiments as long as it is included in the scope of the technological idea substantially same as the present invention. For example, in the sterile filling system of FIG. 6, the mist introducing device 54 and the washing device 57 may be disposed adjacently with the air rinsing device 56 omitted. The decomposing agent supply may be executed when it is needed, and the decomposing agent supply is not the essential item in the present invention. The sterilizing agent is not limited to the hydrogen peroxide, and various kinds of sterilizing agents may be used The sterilizing subject article is not limited to the bottle, but various kinds of food containers such as a cup, a cap and a pouch may be sterilized by the present invention. In addition to the food containers, as long as there is the need of sterilization, the present invention may be utilized.

### EXAMPLES

### Example 1

A 500 ml capacity PET bottle was sterilized according to the procedure of the first embodiment. The specific sterilization procedure was as follows.

(1) The hot air of the temperature at the nozzle opening of 105 to 125° C. was introduced into the bottle from a nozzle having a 10 mm inner diameter by a 0.5 m³/min air amount. At the time, a 35% hydrogen peroxide was vaporized by a ratio of 80 to 400 g per minute so as to produce the sterilizing agent mists, and the same was mixed with the hot air and introduced into the bottle.

(2) Thereafter, the hot air supply was stopped, and 0.5 to 3.5 seconds thereafter, the hot air was blown to the inside of the bottle in the same condition as in the preliminary heating (however, the mists are not mixed) for 1 second.

(3) The hot air supply was stopped, and 1 second thereafter, 1 ml of a 500 U/ml catalase aqueous solution as sprayed to the inside of the bottle.

(4) After 5 seconds, the bottle was inverted, and the sterilized water heated to 70° C. was jetted from a nozzle having a 6 mm inner diameter into the bottle by a 8.5 l (liters)/min for 3 seconds so as to wash the inside of the bottle.

As the catalase aqueous solution, 1% aqueous solution (500 U/ml) of “ASK 50” produced by Mitsubishi Gas Chemical Corp. was used. As to the method of supplying to the bottle, after filtrating the aqueous solution with a membrane filter, the aqueous solution after the filtration was guided to a preliminarily sterilized spray nozzle so as to be jetted into the bottle.

As a result of Example 1, the residual concentration of the hydrogen peroxide was not higher than the detection limit

### Example 2

Based on the second embodiment, the sterilization test was executed with a 500 ml (milliliter) capacity PET bottle provided as the subject while changing the detail conditions for confirming the sterilizing effect. The sterilizing effect evaluation method was as follows.

With 10³, 10⁴, 10⁵ pieces of bacillus subtilis spores each adhered onto five PET bottles, the sterilizing process was executed. A toriputosoibuiyon culture medium was allotted into each bottle after the process sterilely for evaluating whether or not the sterilizing property is effective from the culturing state of the germs in each bottle. According to the statistic method using the MPN (most probable number) method, the number of the surviving germs is presumed in each bottle, and the logarithm value of the number of the adhered germs before the sterilizing process and the number of the surviving germs was calculated by the following formula so as to evaluate the sterilizing effect.

Sterilizing effect=Log(number of adhered germs/number of surviving germs)

(1) Sterilizing Test 1

First, for evaluating the influence of the hydrogen peroxide gas concentration to the sterilizing effect, the following sterilizing test was executed.

While changing the concentration of the hydrogen peroxide, the hot air mixed with the hydrogen peroxide mists was introduced to the 500 ml capacity PET bottle for confirming the sterilizing effect. The hot air temperature was 100° C., the flow rate was 280 L/minute, and the hot air blowing-in time was 3.3 seconds. The relationship between the set hydrogen peroxide concentration, the sterilizing effect and the residual hydrogen peroxide concentration was as in the following table.

TABLE 1

| | | Residual hydrogen |
|---|---|---|
| Hydrogen peroxide | | peroxide |
| concentration (mg/L) | Sterilizing effect | concentration |
| 1.4 | 1.4 | 0.1 |
| 2.7 | 4.5 | 0.3 |
| 4.4 | 6.5 | 0.5 |
| 5.5 | 7.5 | 0.7 |

From these results, it is learned that the sterilizing effect can be made higher with a high hydrogen peroxide concentration, however, with a higher concentration, the concentration of the residual hydrogen peroxide is made higher as well. Since the sterilizing effect depends on also the hot air blowing-in time, a desired sterilizing effect can be obtained by optionally selecting the combination of the hydrogen peroxide concentration and the hot air blowing-in time.

(2) Sterilizing Test 2

For evaluating the influence of the hot air flow rate at the time of supplying the hydrogen peroxide mists to the sterilizing effect, the sterilizing process was executed with the hot air flow rate changed as in Table 2-1. Moreover, the results of the conventional sterilizing method of separately executing the preliminary heating with the hot air and the sterilizing agent mist supply executed as a comparative example are shown in Table 2-2. The hydrogen peroxide mist blowing time in Table 2-1 was 3.3 seconds.

TABLE 2-1

| | Hydrogen peroxide | |
|---|---|---|
| Flow rate (m³/min.) | concentration | Sterilizing effect |
| 0.78 | 4.1 | >7.3 |
| 0.66 | 4.1 | >7.3 |
| 0.33 | 4.1 | >7.3 |
| 0.28 | 4.1 | >7.3 |
| 0.22 | 4.1 | >7.3 |
| 0.11 | 4.1 | 5.4 |

TABLE 2-2

| | Hydrogen peroxide | |
|---|---|---|
| Flow rate (m³/min.) | adhering amount | Sterilizing effect |
| 0.78 | 43 μL | 6.9 |
| 0.56 | 43 μL | 5.5 |
| 0.33 | 43 μL | <4 |

As it is apparent from Table 2-1, if the hydrogen peroxide concentration is constant, the sterilizing effect is not significantly influenced even in the case where the hot air flow rate is changed. As it is shown in Table 2-2, according to the conventional sterilizing method, the sterilizing effect is changed apparently by the hot air flow rate at the time of the preliminary heating so that the superiority of the sterilizing method of the present invention is apparent.

(3) Sterilizing Test 3

For evaluating the influence of the hot air temperature to be mixed with the hydrogen peroxide to the sterilizing effects the sterilizing process was executed with the hot air temperature changed as shown in Table 3-1. The hot air flow rate was set at 0.28 m³/minute, and the hydrogen peroxide concentration was 4.1 mg/L. Moreover, as a comparative example, the results of executing the sterilization with the hot air temperature changed in the conventional sterilizing method of separately executing the preliminary heating with the hot air and the sterilizing agent mist supply are shown in Table 3-2.

TABLE 3-1

| Hot air temperature | Hydrogen peroxide | |
|---|---|---|
| (° C.) | concentration (mg/L) | Sterilizing effect |
| 135 | 4.1 | 6.9 |
| 120 | 4.1 | 7.3 |
| 100 | 4.1 | 6.3 |
| 80 | 4.1 | 6.3 |

TABLE 3-2

| Hot air temperature | Hydrogen peroxide | |
|---|---|---|
| (° C.) | adhering amount | Sterilizing effect |
| 150 | 43 μL | 6.1 |
| 110 | 43 μL | <3.7 |

As it is apparent from Table 3-1, in the case where the hydrogen peroxide concentration is 4.1 mg/L, it is learned that the sterilizing effect is not influenced by the temperature if the hot air temperature is 80° C. or higher in contrast, in the comparative example, the sterilizing effect differs by the preliminary temperature rise temperature even at the high temperature range of 110° C. or higher without the sterilizing effect difference in the present invention.

As heretofore mentioned, although the sterilizing effect is influenced significantly by the bottle temperature at the time of the preliminary heating and the hot air flow rate in the conventional sterilizing method, the influence of the hot air temperature and the flow rate to the sterilizing effect can be made extremely smaller in the present invention.

### INDUSTRIAL APPLICABILITY

According to the sterilizing method and the sterilizing apparatus of the present invention, the sterilizing agent mists can be introduced evenly to every corner of the inside of the article by utilizing the hot air supplied into the article. Since the temperature in the bottle can be maintained constantly by continuously supplying the heat into the bottle by the hot air while introduction of the mists, even if the hot air temperature or flow rate is set at a low level, the even and sufficient sterilizing effect can be obtained. That is, according to the present invention, since the sterilizing condition of introducing the sterilizing agent mists to the inside of the article under a constant temperature for a certain period can easily be realized, the even and excellent sterilizing effect can be obtained certainly in the case of sterilizing the various kinds of the article.

1. A method for sterilizing an article, comprising a process of mixing mists of a sterilizing agent with hot air, and a process of supplying the hot air mixed with the mists into the inside of the article.

2. The sterilizing method according to claim 1, comprising a process of washing the inside of the article with a washing agent after supplying the hot air mixed with the mists into the inside of the article.

3. The sterilizing method according to claim 1, comprising: a process of discharging the air containing the mists inside the article by blowing in the sterilized air into the inside of the article after supplying the hot air mixed with the mists into the inside of the article; and a process of washing the inside of the article with a washing agent after discharging the air including the mists.

4. The sterilizing method according to claim 1, wherein the hot air is blown in from a nozzle by inserting the nozzle into the inside of the article, and a guiding member is disposed around the nozzle outside the article so that the hot air discharged from the article is guided to the outer surface side of the article by the guiding member in the process of supplying the hot air mixed with the mists.

5. The sterilizing method according to claim 2 or 3, wherein the washing agent is heated and supplied to the inside of the article in the washing process.

6. The sterilizing method according to claim 3, wherein the sterilized air is heated and blown into the article in the process of discharging the air including the mists.

7. The sterilizing method according to any one of claims 1 to 6, wherein the article is a food container.

8. The sterilizing method according to any one of claims 1 to 7, wherein the article has a bottle shape.

9. A sterilizing apparatus comprising a hot air supplying device for supplying hot air to the inside of an article, and a mist generating device for producing mists of a sterilizing agent, wherein the mists produced by the mist generating device can be introduced to the inside of the article while being mixed with the hot air supplied by the hot air supplying device.

10. The sterilizing apparatus according to claim 9, comprising a washing device for supplying a washing agent to the inside of the article.

11. The sterilizing apparatus according to claim 9, comprising: an air rinsing device for discharging the air including the mists inside the article by blowing in the sterilized air to the inside of the article; and a washing device for supplying a washing agent to the inside of the article.

12. The sterilizing apparatus according to claim 9, wherein the hot air supplying device comprises: a nozzle to be inserted to the inside of the article for blowing in the hot air into the article; and a guiding member provided so as to surround the nozzle at the outside of the article for guiding the hot air discharged from the article to the outer surface side of the article.

13. The sterilizing apparatus according to claim 10 or 11, wherein the washing device heats the washing agent and supplies the same to the inside of the article.

14. The sterilizing apparatus according to claim 11, wherein the air rinsing device heats the sterilized air and blows in the same into the article.

15. The sterilizing apparatus according to any one of claims 9 to 14, configured to sterilize a food container as the article.

16. The sterilizing apparatus according to anyone of claims 9 to 15, provided with the premise that the article has a bottle shape.
